(19) United States
(12) Patent Application Publication
(10) Pub. No.: US 2004/0030258 A1
(43) Pub. Date: Feb. 12, 2004

(54) Sensor assembly for monitoring an infant brain

(76) Inventors: Christopher Edward Williams, Auckland (NZ); Terrie Eleanor Inder, Key East (AU); Elizabeth Mary Buckland, Oxford (NZ)

Correspondence Address: Blakelu Sokoloff Taylor & Zafman, Seventh Floor, 12400 Wilshire Boulevard, Los Angeles, CA, 90025-1026, US

(21) Appl. No.: 10/398,729

(22) PCT Filed: Oct. 9, 2001
(86) PCT No.: PCT/NZ01/00218

(30) Foreign Application Priority Data
Oct. 9, 2000 (NZ) 507384

(51) Int. Cl. A61B 5/0476

(52) U.S. Cl. 600/544

(57) ABSTRACT

A flexible, conformable, sensor assembly is provided, including an electrode array especially adapted for stable, long-term recording of EEG signals from a pre-term or neonatal infant in intensive care. A kit or sterile pack includes guidance for placement of the electrodes over a designated area of the infant's brain, an area likely to be injured. The sensor assembly includes a left-side and a right-side flexible strip bearing at least electrodes and optional temperature, motion, and optical sensors provide for the monitoring of an extended range of parameters including aspects of cerebral perfusion and metabolism. Optional impedance measurements provide an indication of neuronal swelling. Stable performance over from three days to about a week is intended so that progress, effects of treatment, and outcome can be considered.

### FIELD

This invention relates to assemblies of sensors including skin recording electrodes compatible with electroencephalography and in particular to assemblies adapted to be used over period of time with very young babies including those born prematurely.

### BACKGROUND

To be a premature baby is to be at considerable risk of brain injury. (5-10% of the very low body weight (VLBW) set (n=50,000 per annum, in the USA) have serious neurological difficulties in later life, and up to 50% have subclinical defects perhaps only apparent as learning difficulties). A variety of vascular causes are implicated in the two common syndromes, intra-ventricular hemorrhage (IVH) and periventricular leucodystrophy (PVL). 15% of the VLBW set suffer IVH. See Volpe, J Neurology of the Newborn, 4th ed Saunders at p 428. Both syndromes can result in unilateral or bilateral defects in a similar topographical area of the brain. White matter injury just dorsal and lateral to the external angle of the lateral ventricle is a characteristic parenchymal lesion associated with IVH. The area of interest is alternatively stated to be “the arterial border zone between the occipital and parietal cortex (the middle cerebral artery), and “particularly around the posterior horns of the lateral ventricles”.

Those common syndromes are separate from hypoxic-ischemic encephalopathy seen in term or near-term infants which is attributed to the birth process itself and which occurs at about one tenth the frequency of the IVH/PVL group.

The inventor's group is active in “neuronal rescue” (as per Scheepens et al, WO 00/13650) and has constructed a “brain damage monitor BDM)” (see Williams, U.S. Pat. No. 5,807,270, Williams et al, WO98/57139, and Williams, WO99/15067), capable of making real-time displays of trends of neural function occurring over periods of from minutes to weeks. Several electrical measures of brain function are used therein, including transcranial impedance and in particular various derivatives of EEG signals principally power content, and spectral distribution, seeking to identify a relative loss of higher frequency components. The inventors have a requirement to monitor the electroencephalogram of a particular type of patient (pre-term infants in critical care) with the “brain damage monitor” (BDM) in one version or another over a long period (such as several days or more), in order to evaluate neuronal injury and brain lesions. Many such lesions (though not all) manifest themselves as adverse outcomes by 18 months of age.

The BDM makes use of trends recorded over extended periods in order to report on progress of an injury and the effect of treatment on the lesion. Any EEG comprises a very small low-frequency AC signal, easily lost within intrinsic noise and/or external interference. Changes in the electrode-head coupling over a period of time may introduce artefacts and interfere with clinical judgements. It is clear that altering the “coverage” or the contact of the EEG electrodes is likely to alter the composition of the signal, and thereby mask any clinical changes. It is likely that smaller yet clinically significant details of any long term change within the EEG due to a disease process will be obscured and there is a clear need to be able to “place and forget” a good quality set of electrodes.

The general problem to be solved is to reduce the occurrence of brain damage in neonates by assisting in the management and an understanding of brain injury, including the services of detecting correlates of injury and of following the progress of any damage including effects of any treatment.

The group desires to provide a stable input electrode assembly for the head of a baby—pre-term or term or after, so that a continuous record of correlates of brain injury can be obtained for the above purpose. Hence the particular problem to be solved herein is to provide an assembly including at least a set of skin electrodes of the electroencephalography (EEG) type which set is capable of providing consistent signals over lengthy periods of time when used with very young babies including pre-term babies.

Although some prior art electrode assemblies are made on a flexible substrate, all are relatively large, usually include rigid parts and none describe adaptations that can solve the above problem; including the related problem that premature babies have very thin, sensitive skin with a very thin protective layer of partially keratinised squamous epithelium. Yet there is a requirement for continuous use of such electrodes on the head of a baby within an incubator. At times the baby may lie upon the electrode set, so rigidity of parts is undesirable. Lead dress is also important, particularly if a bonnet for assisted respiration is used.

A number of published patents describe electrode sets or electrode assemblies based on flexible printed-circuit board technology for application in a predetermined layout to the skin in order to collect heart (ECG) or brain (EEG) signals. These include Ayer (U.S. Pat. No. 353,372), Zdrojkowski (U.S. Pat. No. 4,122,843; including a shield behind each electrode), Imran (U.S. Pat. No. 5,327,888), Cram (U.S. Pat. No. 5,772,591) and Bennet et al (U.S. Pat. No. 6,233,472). In particular, U.S. Pat. No. 6,032,064 Devlin et al describes a pre-gelled disposable, self-adhesive three (or more) terminal electrode assembly. Enhancements include a satellite electrode at the end of a waisted strip (U.S. Pat. No. 6,032,064 Devlin), on-board unique identification (U.S. Pat. No. 6,032,064 Devlin and U.S. 5,813,404 Devlin et al, means for avoiding bridging by electrolyte (and consequent shorting out) between electrodes (U.S. Pat. No. 4,082,087 Howson (ribs), U.S. Pat. No. 6,032,064 Devlin et al (pockets for excess)) and an integrated connector with no lead wire U.S. Pat. No. 6,032,064 Devlin et al. Few specify suitability for children or babies. U.S. Pat. No. 5,213,952 Hoch discloses a firm yet flexible belt for holding ECG electrodes securely against an infant's body. WO97/10747 Cleveland describes a wireless EEG system for recording evoked response type EEGs from infants. The electrodes are ordinary ones held down under a bandage or cap. Known EEG electrode sets are quite unsuitable for long-term use on young babies, such as neonates. Both the overall structure including rigid parts, and the aggressive methods used with adults for lowering skin impedance will cause harm. Axelgaard et al (including U.S. Pat. No. 6,263,226) presents a form of easily peelable flexible electrode based on layers of hydrogels having different properties cured so that they hold better to each other and to the flexible conductive surface than they hold to the skin.

Combinations of electrodes on flexible bases with either other sensor devices or inclusion of electronics such as head preamplifiers are uncommon in the patent literature, perhaps because disposability and cost of electronics are incompatible. U.S. Pat. No. 6,259,939 Rogel describes a multi-electrode chest and limb electrode array that includes an ECG transmitter for a wireless connection to the ECG recording means; useful in exercise physiology for example.

### OBJECT

It is an object of this invention to provide a sensor assembly compatible with consistent, long-term use in brain activity recording for young babies, or at least to provide the public with a useful choice.

### STATEMENT OF INVENTION

In a first broad aspect, the invention provides a sensor assembly adapted for placement on a head of a patient (a neonate or infant having an age in the range of about 20 weeks gestational age to about one year after full term) for monitoring, over an extended collection period, the status of the brain of the patient by at least providing for collection of electroencephalography (EEG) type signals from the brain, wherein the sensor assembly includes a first and a second flexible strip connected through a flexible cable to a connector; each strip being comprised of a matrix having a low thickness, each strip having a first surface capable of being applied to the skin of the head and then of conforming to the shape of the head, and each flexible strip of the sensor assembly includes at least two flexible electrodes on the first surface, each electrode being connected by flexible wiring through a connecting lead to a connector, and spaced apart so as to be capable of collecting electrical activity from an area likely to exhibit brain injury, and each flexible strip of the sensor assembly includes at least one releasable adhesive area, capable of resisting forces tending to dislodge the strip during exposure to a warm humid environment, yet capable of being peeled off after a period, without damage to the skin of the subject, so that the electrode assembly is held in place during the collection period, and each flexible strip of the sensor assembly is accompanied by orientation information capable of guiding a person in the process of correctly placing the strip upon the head of the patient so that the strip is placed consistently over an area likely to exhibit brain injury in an intended position and orientation.

In a first related aspect, each flexible electrode is a conductive area, supported on the matrix, and provided with a conductive hydrogel coating.

In a subsidiary aspect, each flexible conductive area is comprised of a plurality of connected sections thereby providing greater flexibility for conforming to the shape of the head.

Preferably the sensor assembly is optimised in terms of flexibility and dimensions for use with neonates in the range of 22-34 weeks, alternatively for use with those neonates in the “very low birth weight” class under 1500 grams.

In a second related aspect, each sensor assembly also includes at least one sensor capable of detecting signals other than the EEG; the at least one sensor being selected from the range of: motion sensors capable of detecting extrinsic or intrinsic patient movement, temperature sensors capable of measuring the surface temperature of the patient, optical sensors capable of measuring the optical density of the underlying tissues (and hence capable of detecting hemorrhage), pulse sensors capable of measuring a pulsatile flow of blood in the underlying tissues, colour-sensitive sensors capable of sensing the amount of oxygenation of blood in the underlying tissues, and colour-sensitive sensors capable of sensing metabolic indicators within brain tissue having characteristic absorbtion spectra including cytochromes.

In a third related aspect, a further flexible lead is provided to a reference electrode capable of being attached to the patient.

In a fourth related aspect, orientation information for placing each flexible strip is based on the “10/20” standard EEG electrode placement system

In a subsidiary aspect, the orientation information comprises guide strips reversibly attached to each sensor assembly, the guide strips providing guidance to align each flexible strip (a) horizontally in relation to an erect head, (b) at about 40% of the distance from the anterior fontanelle to the corresponding ear canal of the patient, and (c) with inter-electrode spacing being preferably about 20% of the distance from the inion to the nasion. Alternatively these guide strips lead a user to place the electrodes on or about the C3,C4, P3, and P4 sites according to the 10-20 international system for the collection of EEG.

In a fifth related aspect, orientation information for each flexible strip guides the placement of each strip over an injury-prone portion of the brain slightly dorsal and lateral to the posterior horn of the lateral ventricle.

In a subsidiary aspect, the orientation information comprises means to (a) determine the position upon the head of the patient of a first line joining the corresponding ear canal to the posterior fontanelle, (b) means to similarly determine a second line from the anterior fontanelle and forming an angle (the angle anterior fontanelle—meeting point—posterior fontanelle) with the first line on the posterior foramen side, the intersection thereby determining the position of a first electrode and (c) the second electrode is to lie on the second line.

Preferably the angle is between 90 and 130 degrees; more preferably the angle is about 110 degrees

Preferably a succession of slanting lines is printed on each flexible strip in order to indicate different displacements for infants of different ages.

In a second broad aspect the invention provides a disposable kit including a sensor assembly as previously described in this section, wherein the kit holds supporting materials, including skin preparation materials, electrode assembly location means, and cable tie-down strips.

Preferably the kit of parts includes a sensor assembly including an electrode array, a conductive gel such as hydrogel connecting wire and connector (all as previously described in this section), retaining tape, skin preparation materials, and a set of instructions for use, so that a non-expert can place the electrodes on the head of a patient.

Preferably the kit also provides hand coverings capable of being peeled away from surfaces coated with hydrogel.

Optionally the pack also includes a bonnet capable of covering the patient's head and covering the electrodes. Optionally also the bonnet is electrically conductive and capable of being grounded so as to act as a shield.

Preferably the pack is capable of being sterilised so that the risk of introduction of pathogens into an incubator can be minimised.

In a third broad aspect this invention provides a cranial electrode array ready for use with the brain status monitor for babies, wherein the electrode array comprises more than one skin electrode, each skin electrode is provided with a non-irritant gel capable in use of providing an effective coupling between the metal electrode and the underlying skin of the baby, the skin electrodes are fixed beneath a support surface comprised of a soft, flexible non-conductive material so that the relative position of each electrode to others is predefined and the dimensions of the support surface are selected so as to match the electrode spacings with intracranial structures to be monitored; so that the brain damage monitor can be used to assess the state of the brain of a prematurely born infant which brain may have undergone trauma or the like.

A preferred non-irritant gel comprises “hydrogel”.

Preferably the invention is constructed on a “flexible printed circuit” base. More preferably the base is a soft plastics material capable of conforming to a spheroidal surface.

Preferably the flexible printed circuit base is narrowed at regions between electrodes in order to permit more twisting of the base than would otherwise be possible.

Preferably there are three electrodes; a pair of active electrodes and a reference electrode; in normal use the active electrodes being intended to be connected to inputs of an amplifier and the reference electrode being intended be connected to a reference input of an amplifier forming a front stage of a brain damage monitor.

Optionally a shield layer of conductive material is placed above the support surface.

Preferably extendible wires are embedded in the support surface.

Preferably the flexible wire emerges from the electrode array in a direction such that the wires pass behind the ears and away from the head.

Preferably the connector includes information-carrying means pertaining to the characteristics of the sensor array to which it is attached.

Optionally there is a separate connector for the left side and a separate connector for the right side and optionally there is a separate cable for each separate connector.

In a fourth broad aspect the invention also includes optically active leads or fibres so that incident, reflected, and/or transmitted light may be carried to and from the head of the patient; for example in order to examine coloration (as in relation to peripheral and/or CNS perfusion) and/or enzyme activity.

In a related aspect, fibre optic techniques may be used to measure temperature and/or stress or relative motion within the electrode array.

### PREFERRED EMBODIMENT

The description of the invention to be provided herein, and the illustrative drawings, are given purely by way of example and are not to be taken in any way as limiting the scope or extent of the invention.

### DRAWINGS

FIG. 1: skin-side surface view of an electrode array (with connector) according to the invention.

FIG. 2: section through the distal end of an electrode array according to the invention.

FIG. 3: skin-side surface view of a more flexible electrode array laid down on a flexible printed-circuit substrate.

FIG. 4: skin-side surface view of an electrode array including optical sensors and a thermistor, according to the invention.

FIG. 5: section through the distal end of an electrode array including one or more optical fibres for use in sensing metabolic parameters.

FIG. 6: section through a head showing likely light paths between applied electrodes.

FIG. 7a: One preferred position for electrodes to monitor brain function superimposed on a drawing of a 37 week baby's head, with landmarks.

FIG. 7b: Prior art: illustration of parasagittal cerebral injury distribution in relation to major cerebral arteries. (Volpe, FIG. 8-7)

FIG. 8: Another preferred position for electrodes to monitor brain function superimposed on a drawing of a 37 week baby's head, with landmarks.

FIG. 9: Circuit diagram of an example electrode set, including connector assembly, cable, cranial electrodes, movement sensor, and grounding electrode.

FIG. 10: (as a,b,c,d and e) Sensor assemblies for monitoring brain function including overlaid markings to point to landmarks.

FIG. 11: (as a and b) Another type of sensor assembly, including overlaid markings to point to landmarks.

### EXAMPLE 1

The cranium of a pre-term infant or neonate has a circumference of very approximately 220-320 mm. The electrode array within the sensor assembly of this invention is adapted in size to match the disposition of intracranial structures to be monitored with the electrode spacings supplied within the electrode array. As indicated in the Background, most neural defects in a pre-term infant or neonate lie in a single (though often bilateral) fairly well defined site (see FIG. 7). The skin of a pre-term infant or neonate has a very thin protective layer of partially keratinised squamous epithelium and hence the rather aggressive means to reduce the barrier imposed by the skin of an adult is not required. In fact, the delicate nature of the skin may be a liability. Practical problems are generally as a result of other medical interventions such as application of oils or petroleum jelly, or use of head coverings.

We prefer to provide each skin electrode, generally of the chlorided silver surface type, with a layer of pre-loaded non-irritant conductive sticky saline gel of the “hydrogel” type. This is capable in use of providing an effective coupling between the metal electrode and the underlying skin of the baby. The hydrogel technology disclosed by, for example, Axelgaard et al (U.S. Pat. No. 6,263,226) is one suitable example. The hydrogel should be easily peelable from the delicate skin, when the electrode is finally removed.

We prefer to provide an array of skin electrodes already fixed beneath (and forming part of) a support surface comprised of a flexible and preferably also soft non-conductive material so that the relative position of each electrode to others is predefined in relation to the most likely site of an underlying lesion. If the electrodes must be replaced the new set ought to provide signals consistent with those from the previous set. (Standard EEG electrodes are freely placeable buttons on flying leads).

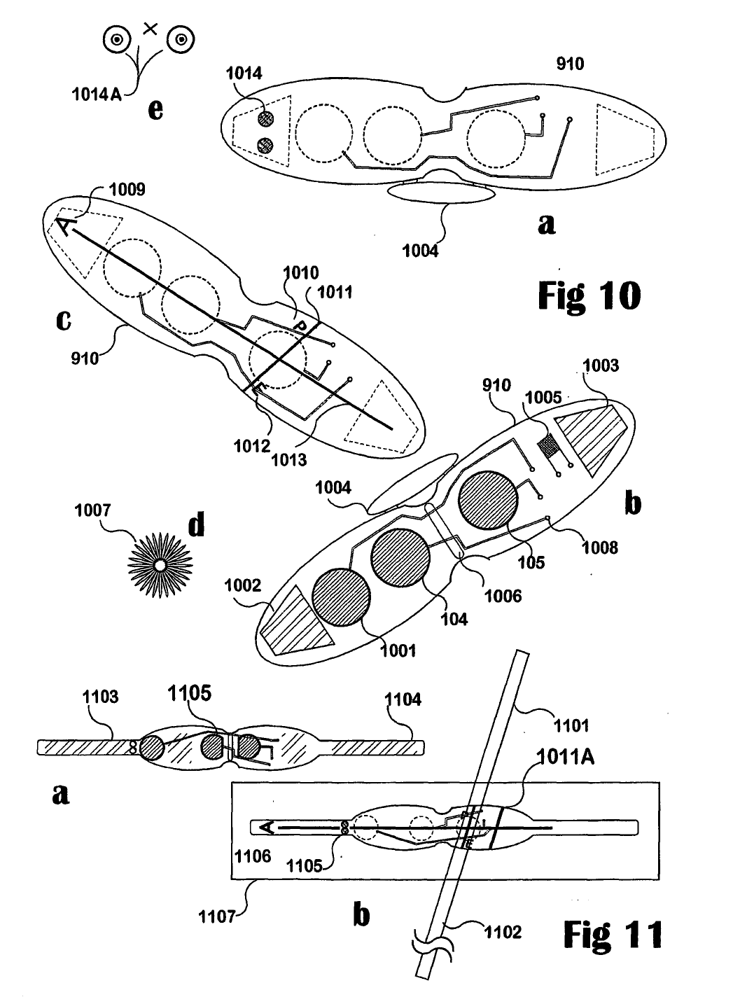

A suitable soft material is a deformable elastomeric plastics material with a biological compatibility rendering it suitable even for implantation. (Some materials include plasticisers and other additives which are toxic if or when they diffuse out from the material). Preferably the substrate is transparent yet coloured. Softness is a relative term; in this case it is to be understood in relation to long-term use in contact with the head of a pre-term infant, at 37-40 deg C. At this time we use a “Mylar” or similar (polyethylene terephthalate) flexible base. This lacks softness but is flexible—it can bend, between electrodes at least, in one plane. The traces on FIG. 4 assume a conventional flexible substrate. The waisting at 301 and 302 in FIG. 3, and in FIGS. 10 and 11 are intended to permit more twisting of the base—simulating distortion in more than one plane—than would otherwise be possible.

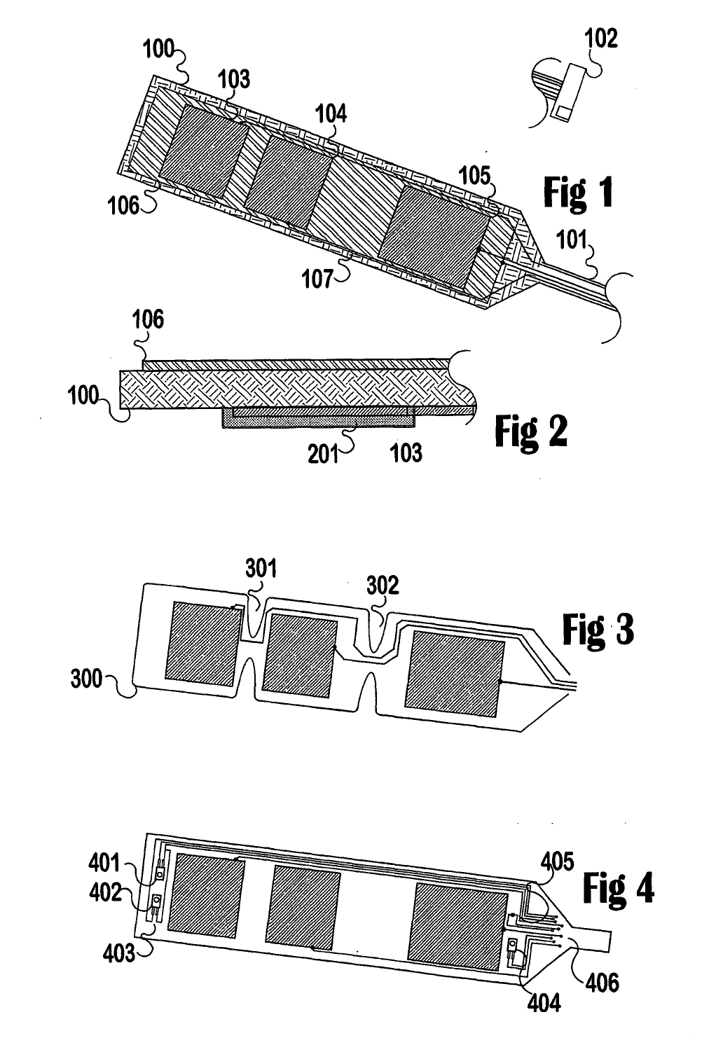

Refer to FIG. 1. This shows a flat strip 100 of a flexible insulating material, bearing three contact-electrode surfaces; 103 and 104 being intended as input electrodes and 105 being a reference electrode. Any wires that may run through a stretchable material should also be made extendible and a “W” layout may be preferable to a coiled layout in terms of interference suppression. We prefer to shield the rear surfaces of each electrode (that is, away from the skin)) with a shield 106 made perhaps of a wire foil or an extensible, perhaps knitted, configuration of wires which is preferably embedded within the flexible material 100, usually as a foil beneath two layers of plastics material). A number of leads are taken from one end of the strip (at 101) and pass through a cable to reach a connector 102 (FIGS. 1 and 9).

The dimensions of the support surface are selected so as to match the electrode spacings with the possibly injured intracranial structures to be monitored. As is well-known in EEG machines, a symmetrical pair of input electrodes 103, 104 are in normal use connected to inputs of an instrumentation amplifier capable of rejecting relatively large signals applied simultaneously to both inputs but of selecting differences between the inputs; using an additional reference electrode 105 which is intended to be connected to a reference input—in effect a zero-voltage or ground input of an amplifier. (In relation to the BDM, recordings are taken from only one input electrode pair or “channel” on each side, rather than repeatedly selecting from many channels). Guard shielding techniques may be used. The closer pair are the active electrodes. The typically 8 mm diameter electrodes are typically placed beneath a straight strip of soft supporting material about 10-12 mm wide, although a bent strip may be preferred. The active side of the strip may include pockets for holding excess hydrogel, as is known in the art.

A preferred electrode construction process is to (1) construct a copper shape (103, 104 or 105) having the dimensions of the or each electrode (using known printed circuit board construction techniques such as photographically etching, or silk-screen printing), (2) apply an insulating coating over all non-electrode areas, (3) print over the area of the or each electrode with a silver conductive ink or paint, (4) apply an electrolysis treatment to each electrode in a saline electrolyte so as to create a chlorided silver active surface, and (5) print a hydrogel coating 201 over the silver.

Given that a flat metal sheet, even though it is thin, is inherently not soft and flexible, we provide a configured electrode that is able to conform to a convex surface beneath. See FIG. 10d, which shows an electrode having an array of radiating petals extending from a central area, like a daisy or a chrysanthemum. Each petal is able to bend slightly independently of any other. This arrangement includes a number of sharp edges of very low height—around each petal edge, and also saves on metal particularly silver. This modification is useful if the electrodes are prepared on a soft and deformable substrate—though not necessary with “Mylar”.

### EXAMPLE 2

One electrode set is likely to have several days of use. Because proper monitoring of brain injury may prevent the baby from suffering permanent brain damage, further active circuitry may be economically justified within a sensor assembly. For BDM applications, a temperature sensing transducer 405 is an example of an additional feature. There may be clinical justification for applying cooling to the head and a cooling cap (for control of injury-related pathophysiology) is another product of the inventor's team. Similarly, a movement sensing transducer (usually a moving coil (908) or a piezoelectric device) may be embedded in the support surface or in a forehead ground electrode in order to provide extra information about the state of the patient. The EEG signals may be discarded during movement so that movement artefacts do not affect interpretation.

Optionally, blood presence, blood amount, or blood oxygenation sensors, based on optical methods are included with the electrode array, preferably embedded in the support surface. For example a sensor may comprise a set of devices capable of determining the relative proportion of oxygenated haemoglobin in blood beneath it—using bands of visible or infra-red light with a colour difference technique. Two colours may be selected so that for example one is absorbed by unsaturated haemoglobin and the other is absorbed by saturated haemoglobin—so providing data for an oximeter along with a sensor 404. Pulse rate can also be derived from returned light. It has been found that if the sender and receiver of light are more than 60 to 80 mm apart in the same assembly then most of the light reaching the receiver will have been transmitted through the cerebral cortex at least. The skulls of neonates are relatively translucent. It is possible to send and receive light from the same array, or, for use in detecting intraventricular hemorrhages—to send light from one array right through the patient's head to the other.

FIG. 4 illustrates inclusion of embedded optoelectronic devices. 401 and 402 are example light-emitting diodes. The peak wavelength of these devices can be varied by selection of the semiconductor and doping thereof, and the “colour” —usually a near infrared—can be further modified by filtering. In order to prevent a light-pipe effect through the substrate 100 of the electrode array, this may be coloured 403 by the addition of a pigmented filler—carbon, or ferric oxide. Compatible pairs of infra-red (IR) emitters and receivers may be embedded within or attached to the printed-circuit base of the electrode array. Drive currents for diodes 401, 402 might be brief pulses of hundreds of milliamperes which are likely to interfere with the EEG recording—though the BDM is likely to correctly identify the interference and discard it. Use of a ground plane within the flexible electrode will allow active signal processing techniques to be embedded on the flexible strip. The ground plane will act as a shield to prevent noise from interacting with for example an analogue preamplification section.

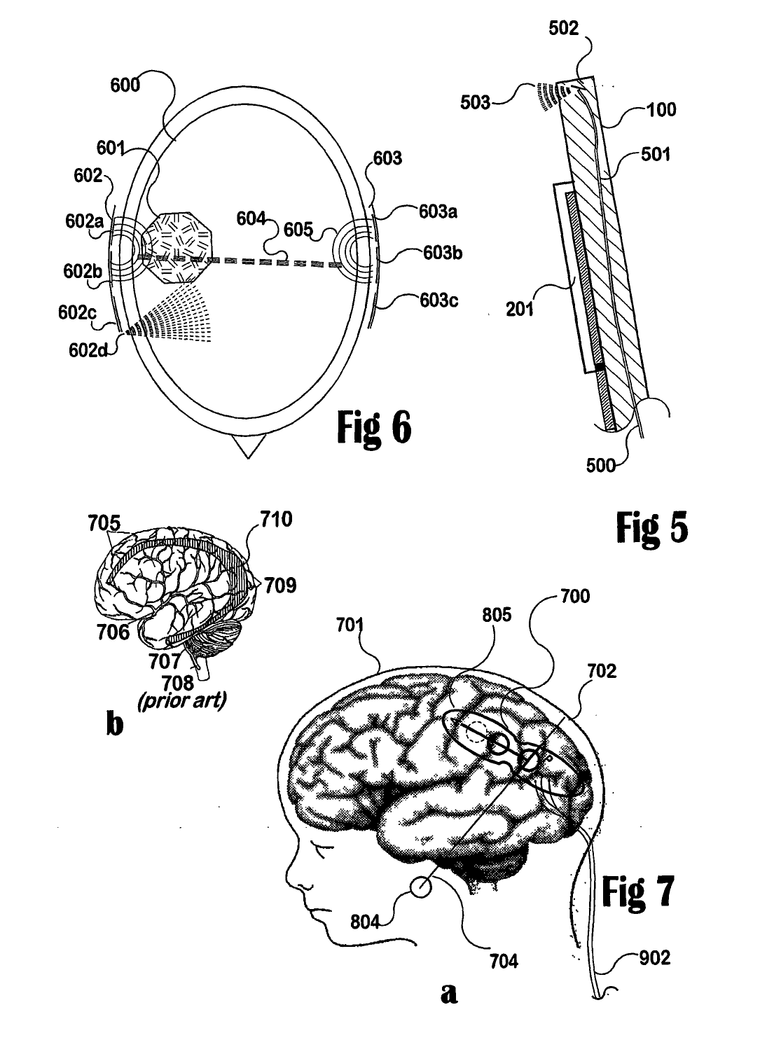

The circuit diagram of FIG. 9 (900) shows two sensor assemblies, 910 and 911; one for each side of an infant's head, joined (in this example) to a single connector 102. In this example, each sensor includes a pair of input electrodes (105 and 105; 104A and 105A). There are carried by a flexible shielded cable 902 preferably about a metre long to connector pins 901 within connector 102. (Note the grouping of some pins into a “R” and a “L” group. It is possible to use separate connectors for each side.) A preferred flexible cable is a screened silicone-coated cable, which is quite flexible and may be sterilised. Shielding (913) may be implemented in many ways such as over the entire cable or selected wires, with actively driven guards and similar, as is known to one skilled in the art. A separate grounding electrode 909 may be used. Additional sensing means illustrated in FIG. 9 include: thermistor 907 for monitoring skin temperature, light source (LED) 401 and light sensor (photo transistor) 404 for monitoring blood-related quantities, and an electromagnetic motion sensor 908 used to indicate when signals should be rejected for motion artefacts. The permanent magnet core is able to shake within the windings hence inducing a current when external motion occurs. The connector 102 also includes an information storage chip 903, connected by DC power leads 904 and 906 and by data leads 905, 905A (assuming a suitable data transfer protocol such as 12C) to the connector. This is used for sensor-specific descriptions and includes thermistor calibration information, electrode type, version (such as which additional sensors are provided), and date of manufacture information loaded during manufacture of the sensor assembly. The BDM is capable of reading and using information from this storage chip 903. This diagram shows only one example of many useful combinations of sensors and does not include the option of optical fibres as in FIG. 5 for example. Nor does the diagram include on-sensor amplifiers or other electronics, nor the intra-matrix screening layer. The electrodes of the sensor assembly may be used without alteration for trans-cranial cerebral impedance measurement and detection of impedance change.

We prefer not to place the connector on the electrode strip itself because the size of the type of connector involved imparts a non-flexible rigid lump to the connector. Lead attachment is by low-temperature soldering, adding only a minimum thickness to the sensor assembly.

The connector pair is adapted to provide for positive engagement of the two parts so that a user can feel a “click” representing a secure connection, and so that the connector pair does not inadvertently come undone. Preferably the engagement is a fluid seal, so that fluid contamination (as by dripping saline) is unlikely to occur, with resulting corruption of the signals; preferably with gold-coated connector surfaces for the sake of low electrical noise. Fibre optics can serve as transducer coupling means and the connector can then also serve to abut fibre optic single filaments or multi-stranded fibres as required. A custom connector can also be designed to prevent inadvertent connection to other equipment which might comprise an electric shock hazard to the baby.

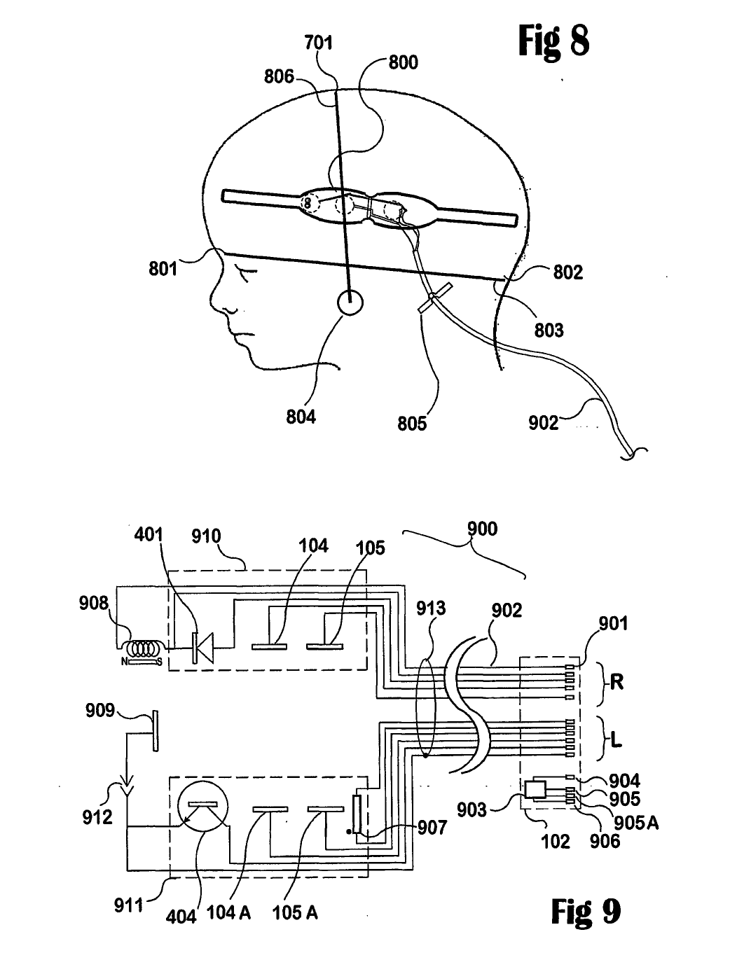

FIG. 10 shows several versions of an improved sensor assembly. Diagram “a” shows an assembly with direction-indicating indicia (see FIGS. 7 or 8) printed on the outside surface; a “nose dots” icon 1014 at the left and an “ear tab” 1004) below. This is for the left side of the head. The right-side assembly would have the ear tab on the opposite side. In case use of two dots to indicate the direction of the nose is inadequate, an alternative is in diagram “e” representing two eyes and the nasion reference point between and slightly above the eyes. Diagram “b” shows the underneath of “a”. Refer also to FIG. 9 for an electric circuit 910 represents the outer edge of the flexible substrate. 104 and 105 are input electrodes and 1001 is an (optional) ground electrode. 1005 is an on-board temperature sensor using an area of temperature-sensitive ink. An improvement to such assemblies is use of a heat-insulating foamy material at least over the thermistor area if not over the entire assembly. 1002 and 1003 are areas of hydrogel, which may be the same conductive hydrogel as used on the electrodes or may be a non-conductive version. Diagram “c” shows the top of a similar electrode adapted for the “third version” of electrode placement (FIG. 7) by having printed upon the outer surface a slanting line 1011 marked with the letters “P” 1010 (to show the direction of the posterior fontanelle) and “E” 1012 (ear canal) for use in the location procedure. The letter “A” 1009 indicates the direction of the anterior fontanelle. Finally, diagram “d” shows one design for a conformable metal electrode in which each “petal” 1007 can bend separately, more easily than can a single metal foil sheet which can bend in only one axis.

FIG. 11 shows a further preferred sensor assembly, wherein the extended lugs 1103 and 1104 of the flexible matrix are provided with a coating of hydrogel (see “a”) to act as a peelable adhesive and because these reach well around the curve of the baby's head this is expected to be a stable variation. In FIG. 11(b) the sensor top view includes the letter “A” (1106) as well as the nose dots icon 1105, hence is compatible with either preferred electrode placement policy. We also show a water-impervious base 1107 onto which the sensor assembly is reversibly adherent, ready to be peeled off for use, and a guide (perhaps paper) strip 1101-1102 which is a built-in line marker for placing between the baby's ear canal and posterior fontanelle. This paper strip has been attached over the electrode using a low-stick adhesive material so that it can be peeled off and thrown away after sensor assembly location is complete.

### EXAMPLE 3

In a neonatal intensive care unit where the BDM is most likely to be used, one BDM on a support is placed beside or attached to an incubator or tent containing the patient for the duration of the study. An optional head stage or preamplifier may be extended from the BDM towards the infant so that interference is reduced and so that the sensor assembly wire is kept reasonably short. The head stage may be the only custom electronics module; the remainder of the BDM may comprise software running in an ordinary personal computer. A disposable kit, including a left and a right set of sensor assemblies having pre-gelled disposable electrodes together with skin preparation materials, labels, and the like provides all the materials needed to connect a neonate's head to a BDM. Instructions are preferably presented on the screen of the BDM but should be repeated on a paper insert or the like within each kit in case the BDM is not available at the point where the electrodes are put in place.

Electrode Placement

At this time there are three variations of electrode placement under trial.

Variation 1: FIG. 8 describes the use of anatomical landmarks for locating a sensor array 800 according to the conventional “C3, C4, P3, P4” EEG electrode sites in the scheme known as “10-20”. This site is located by taking a “latitude” of 40% from the anterior fontanelle 701 down a line 806 to the ear canal 804, and placing the anterior electrode over the line with the posterior electrode on the same latitude, and behind. Under this scheme, electrode center-to-center distance is set at about 20% of the distance along a line 803 joining the inion 802 to the nasion 801 (both are skull landmarks). For small infants, a sensor array having the input electrodes spaced by 2.5 cm (center-to-center) is about right, and for larger babies; 3.5 cm. (Our preferred electrode diameter is 8 mm). Note in FIG. 8 the use of a skin tie-down 805 for the cable 902. Only one side is depicted here although both left and right sides of the head would usually be fitted with a sensor assembly.

Variant 2: uses a similar location but with 2 cm inter-electrode spacing for infants under 26 weeks, 2.5 cm for infants 26-34 weeks, and 3.5 cm for over 35 week infants (or the corresponding weight classifications). It will be appreciated that the gap between the edges of the circular electrodes should be large in order to avoid shorting and to increase the voltage differential. We widen the inter-electrode gap as far as possible perhaps by shaping the electrodes as in 1105, FIG. 11a—and may also include a projecting ridge 1006 in FIG. 10b (perhaps like Howson U.S. Pat. No. 4,082,087).

The third, preferred variant is shown in FIG. 7. Note that it does not correspond to an existing EEG standard. A line 704 is constructed (or visualised) between the ear canal 804 and the posterior fontanelle 702. A sensor assembly 700 for that side of the head is selected and the slanted heavy line printed over one electrode on the sensor assembly is laid upon and in line with the line 704. The sensor assembly is moved along line 704 until the axial line printed on the assembly points at the anterior fontanelle, at which point the sensor assembly is is the correct position and tilt angle. This variant locates the electrodes more precisely over the usual area of injury, as described in the “Background”. FIG. 7b (from Volpe's textbook) shows a drawing of parasagittal cerebral injury distribution 710 occurring in an arc of end-fields of arteries (705: anterior cerebral artery, 706; middle cerebral artery, 707; posterior cerebral artery, 708; basilar artery, and 709; vertebral artery) over the cerebral cortex. The distribution field 710 is in line with the electrode position in this third variation. Because the most frequent site of injury tends to move anteriorly in the older infants The optimal position of the electrodes should be corrected in relation to age (or age-equivalent in terms of weight) of the infant. The slanting line 1011 may be repeated towards the right-hand end (of this example) in the sensor assembly of FIG. 11 in order to indicate optimum positions for different ages. Again, inter-electrode distance may be made smaller for the pre-term babies.

Of course, a baby's head is not a consistent shape nor are the underlying structures necessarily consistently placed, especially when not normal, and specific clinical indications may suggest yet other placements of the sensor arrays of this invention. An astute clinician may realise that the neurological signs shown by a particular baby are atypical and hence may “explore” the head using one of the pair of sensor assemblies; the other perhaps serving as a reference. Nevertheless, screening applications, and re-placement of electrodes on a baby being monitored do tend to require consistent placement.

Disposable Kit for Neonatal Intensive Care

Materials according to the invention are presented in an easy-to-use manner that also minimises mistaken selection (see 2(a) below), so that waste is minimised. Typically the kit would be supplied in an outer well-labelled box with a peel-off cover containing a labelled, sealed, sterile bag labelled also with the usual warning about broken seals. Sterility in the surgical sense is not usually required because no skin penetration is involved, but freedom from any possibly pathogenic micro-organisms is desirable. In practice, there may be deemed to be only one kind of sterility possible. Heat sterilisation may affect hydrogel The kit could be sterilised by ethylene oxide or other suitable and compatible means, as is known in the art.

The preferred kit includes:

1. a rectangle of cloth, of a colour contrasting with the kit contents, for laying out on a table under the contents of the kit when preparing a neonate for monitoring, although optionally each component of the kit may be held ready for use in a pocket in a structure inside the bag.

2. a flexible electrode assembly as previously described including a layer of hydrogel localised to the electrodes, and a ground electrode (which may also include a motion sensor). For hairy scalps, a supply of gel such as “10/20 paste” for pre-term babies and “EC2 electrode cream” (Astro Med) on term or older babies.

a) Skin adhesive should be of limited grip yet able to withstand:

normal hands-on care of babies including intubation and Kangaroo care, without requiring reapplication.

Weight of the baby's head and movement when lying on the electrodes.

Incubator or tent environment; up to 40 dg C and 50-88% relative humidity.

b) The assemblies may be small or large (inter-electrode distance may be varied), and a left and a right side assembly is included. Better adhesion is provided by an elongated strip and provision of waists between electrodes allows further flexibility.

A patch of hydrogel at each end of the strip provides a preferred type of adhesion point.

c) The assembly should have markings to indicate the direction of nose and ear, so that placement is consistent. See FIGS. 10 and 11.

d) Electrode locating indicia (on the sensor assemblies) and electrode locating guide strips.

3. An about 1 metre of flexible lead terminated in a connector; where there is a preference to have the wire or cable dressed so that it comes away from behind the head and downward away from the baby's hands and so that other activities, such as picking up the patient, are minimally affected. Preferred colours are other than the black white and green already used for ECG electrodes.

4. “Hydrogel” strip tie-downs (805, FIG. 8) or similar are included for the cable, so that the cable can be held down onto the skin behind the baby's head until it is directed towards the BDM. The tie-downs may be coloured for convenience. (Strips may be discs or other shapes)

5. skin preparation materials which would be impregnated into at least 3 or 4 sterile cotton bud applicators; using aqueous chlorhexidine (or the like) for the youngest babies with immature skins, and “Nu-prep” for older ones.

a) A few swabs and some sterile water for wiping away skin prep solution.

6. a clear set of instructions for use (see above)

7. a bonnet capable of covering the patient's head and covering the electrodes. This may help in protecting (and hiding) the electrodes, for helping to prevent involuntary movements from taking the electrodes off, and in order to shield the electrodes. Electrical shielding can be provided with a conductive cloth bonnet.

a) preferably the bonnet is compatible with other forms of treatment such as a cooling cap. or a CPAP (continuous positive air pressure) treatment which may include its own bonnet.

8. For electrode location, we prefer

a) either tear-off paper tapes so that the electrodes can be centered on the desired site (as described above and with reference to FIG. 8)

b) or peel-off guide strips, suitably marked. These are particularly useful if the electrode has to be placed by tactile mean—such as under an existing CPAP bonnet.

Hydrogel is known to stick tenaciously to some types of gloves—which may or may not be routinely used in a given neonatal intensive care unit. One approach to overcome this property is to maximise the sizes of borders, covers, and strips so that gloved fingers need not approach the hydrogel-coated parts. Another approach is to supply non-sticky (to hydrogel) plastic gloves or “Teflon” (R) coated finger cots in each kit.

### Commercial Benefits or Advantages

1. Premature babies that survive with brain injury (and there are many) become a significant expense on society to say nothing of distress caused to parents, and the invention is directed towards reducing those effects or at least towards predicting outcome.

2. Electrode assemblies according to the invention are easier to apply in a consistent manner to the head of a small baby, and then are more likely to remain in place and thereby provide consistent EEG type signals from the head of a pre-term infant patient to a brain damage/brain status monitor (BDM) over an extended period, and thereby support the objectives of the BDM.

3. Ease of use for nurses (time spent in placement of electrodes is reduced, tactile-only placement is possible, and replacement is less frequent).

4. The electrodes as described, having for example no hard connectors included, are less likely to cause irritation, abrasions or other trauma to the vulnerable head of a pre-term infant for whom any unnecessary handling presents a risk.

5. Sensor assemblies also including optical and temperature enhancements can collect an increased amount of information for building up clinical experience, as well as for assisting the patient under study, without additional attachments.

6. Provision of a dual-channel system (left and right sensor assemblies) allows exploration of the head of an unusual patient with one assembly, perhaps leaving the other assembly over a “reference area”. This helps in clinical understanding.

7. Repeatable screening programmes can be carried out, such as a routine check for signs indicating injury likely to lead to cerebral palsy later on.

8. The same principles are also extremely helpful for the long-term use of EEG-like electrodes on older patients. In particular, the equipment may be used in older children with hydro-cephalus during the repeated procedures intended to set the intracranial pressure correctly while providing a drain. Too much pressure ininbits neural activity and this can be detected by the BDM.

Finally, it will be understood that the scope of this invention as described and/or illustrated within this provisional specification is not limited to the preferred embodiments described herein. Those skilled in the art will appreciate that various modifications, additions, and substitutions are possible without departing from the scope and spirit of the invention as set forth in the following claims.

1. A sensor assembly adapted for placement on a head of a patient (a neonate or infant having an age in the range of about 20 weeks gestational age to about one year after full term) for monitoring, over an extended collection period, the status of the brain of the patient by at least providing for collection of electroencephalography (EEG) type signals from the brain, characterised in that
a) the sensor assembly includes a first and a second flexible strip each connected through a flexible cable to a connector; each strip being comprised of a matrix having a low thickness, each strip having a first surface capable of being applied to the skin of the head and then of conforming to the shape of the head, and
b) each flexible strip of the sensor assembly includes at least two flexible electrodes on the first surface, each electrode being connected by flexible wiring through a connecting lead to a connector, and spaced apart so as to be capable of collecting electrical activity from an area likely to exhibit brain injury, and
c) each flexible strip of the sensor assembly includes at least one releasable adhesive area, capable of resisting forces tending to dislodge the strip during exposure to a warm humid environment, yet capable of being peeled off after a period, without damage to the skin of the subject, so that the electrode assembly is held in place during the collection period, and
d) each flexible strip of the sensor assembly is accompanied by orientation information capable of guiding a person in the process of correctly placing the strip upon the head of the patient so that the strip is placed consistently over an area likely to exhibit brain injury in an intended position and orientation, in a patient of the above range of age or weight.

2. A sensor assembly as claimed in claim 1, characterised in that the sensor assembly is optimised in terms of flexibility and dimensions for use with neonates in the range of 22-34 weeks.

3. An electrode for a sensor assembly as claimed in claim 1, characterised in that each flexible electrode is a conductive area, supported upon the first surface of the strip, and provided with a conductive hydrogel coating.

4. An electrode as claimed in claim 3, characterised in that the conductive area is comprised of a plurality of connected sections thereby providing greater flexibility for conforming to the shape of the head

5. A sensor assembly as claimed in claim 1, characterised in that the sensor assembly also includes at least one sensor capable of detecting signals other than electrical activity of the brain; the at least one sensor being selected from the range of:
a) motion sensors capable of detecting extrinsic or intrinsic patient movement,
b) temperature sensors capable of measuring the surface temperature of the patient,
c) optical sensors capable of measuring the optical density of the underlying tissues (and hence capable of detecting hemorrhage),
d) pulse oximetry sensors capable of measuring a pulsatile flow of blood in the underlying tissues, and of sensing the amount of oxygenation of blood in the underlying tissues, and
e) optical sensors employing restricted wavelengths of light, capable of sensing metabolic indicators within brain tissue having characteristic absorbtion spectra.

6. A sensor assembly as claimed in claim 1, characterised in that a further flexible lead is provided to a reference electrode capable of being attached to the patient.

7. A sensor assembly as claimed in claim 1, characterised in that orientation information included for placing each flexible strip is according to the “10/20” standard EEG electrode siting information for electrodes.

8. A sensor assembly as claimed in claim 7, characterised in that the orientation information comprises one or more guide strips reversibly attached to each sensor assembly, the guide strips providing guidance to align each flexible strip (a) horizontally in relation to an erect head, and (b) at about 40% of the distance from the anterior fontanelle to the corresponding ear canal of the patient.

9. A sensor assembly as claimed in claim 1, characterised in that the orientation information for each flexible strip guides the placement of each strip over the parasagittal area of the brain lying slightly dorsal and lateral to the posterior horn of the lateral ventricle.

10. A sensor assembly as claimed in claim 9, characterised in that the orientation information comprises means to (a) determine the position upon the head of the patient of a first line joining the corresponding ear canal to the posterior fontanelle, (b) means to similarly determine a second line from the anterior fontanelle meeting the first line at a meeting point, the intersection thereby determining the position of a first electrode and (c) the second electrode is to lie on the second line.

11. A sensor assembly as claimed in claim 10, characterised in that the angle (anterior fontanelle—meeting point—posterior fontanelle) is between 90 and 130 degrees.

12. A sensor assembly as claimed in claim 11, characterised in that the angle is about 110 degrees.

13. A sensor assembly as claimed in claim 1, characterised in that the connector includes solid-state information storage means capable of being loaded with, holding, and from time to time supplying a description of the characteristics of the sensor assembly.

14. A disposable kit including at least one sensor assembly as claimed in claim 1, characterised in that the kit holds supporting materials, including skin preparation materials, electrode assembly location means, and cable tie-down strips

15. A disposable kit as claimed in claim 14, characterised in that the kit also provides hand coverings capable of being peeled away from surfaces coated with hydrogel.
